(19) United States
(12) Patent Application Publication
(10) Pub. No.: US 2007/0282001 A1
(43) Pub. Date: Dec. 6, 2007

(54) Materials And Methods For Improving Shellfish Health, Immunity And Growth

(76) Inventor: Francis Chi, Kowloon (HK)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/630,454

(22) PCT Filed: Jun. 28, 2005
(86) PCT No.: PCT/EP05/06948

§ 371 Date: Jul. 26, 2007

Related U.S. Application Data
(60) Provisional application No. 60/584,574, filed on Jun. 30, 2004.
(60) Provisional application No. 60/646,284, filed on Jan. 24, 2005.

(51) Int. Cl. A61K 31/145; A61K 31/197

(52) U.S. Cl. 514/563; 514/665

(57) ABSTRACT

The subject invention provides methods for improving shellfish health. In specific embodiments, the invention provides methods for accelerating and/or augmenting shellfish growth; improving immunity; and enhancing fertility in shellfish. To do so, the present invention provides materials and methods for administering a cysteamine compound to shellfish.

### BACKGROUND OF THE INVENTION

The world's population is increasing rapidly, causing a concurrent increase in demand for seafood. Moreover, recent studies demonstrating the beneficial properties of shellfish have added to the demand (see Takezaki, T. et al., “Diet and lung cancer risk from a 14-year population-based prospective study in Japan: with special reference to fish consumption,” Nutr Cancer, 45(2):160-7 (2003); and Su, X. et al., “Omega-3 polyunsaturated fatty acid content in different edible portions of commercial scallop,” Asia Pac J Clin Nutr., 12 Suppl:S63 (2003)). A variety of methods have developed in attempts to bridge the gap between the supply and demand of seafood. For example, many different shellfish species are now produced in aquacultures. Some species are cultured during the complete life cycle whereas others are cultured from wild-harvested seed. Still others are raised in hatcheries and released to open water for later harvesting.

Unfortunately, the cost in time and operation for producing marketable sized shellfish using the methods described above is very high. For example, start-up costs alone for establishing an oyster farm in Alaska can begin in the tens of thousands and rapidly ratchet up to hundreds of thousands as the size of the farm increases.

The growth cycle for most shellfish is lengthy in time. For abalone and lobsters, for example, several years are often necessary for development and growth before obtaining a desirable size for consumption. Therefore, there is a need for shortening the growth cycle for shellfish and augmenting growth to more rapidly provide marketable shellfish to the consumer.

Diseases and contaminated water are a threat to any shellfish aquaculture, hatchery, or farm. Most diseases in shellfish are caused by opportunistic pathogens, i.e., bacteria that cause disease in shellfish that are weakened or stressed. Microscopic marine organisms, such as the one-celled dinoflagellates that produce a CONFIRMATION COPY toxin called paralytic shellfish poison (PSP), can accumulate in exposed shellfish and be harmful to the end-consumer.

Vaccines and antibiotics are two available methods used to protect shellfish against diseases. The use of drugs (i.e., antibiotics, vaccines, etc.), which is expensive, has to be considerably reduced in aquaculture to avoid environmental hazards and the risk of the development of resistance. Therefore, there is a constant need for enhancing the immunogenicity of shellfish.

Cysteamine is a depleting agent of the growth inhibitor somatostatin, which can increase growth and longevity in vertebrates by promoting growth hormone secretion from the pituitary. However, shrimp are invertebrate animals, which have a different metabolism and endocrine system from that of the vertebrate animal. Thus, whether cysteamine can promote the growth of shrimp, let alone improve the health of shellfish, is still a question. Insofar as is known, cysteamine compounds have not been previously reported as being useful for improving the health of shellfish.

### BRIEF SUMMARY OF THE INVENTION

The subject invention provides materials and methods for improving the health of shellfish. In particular, the present invention concerns materials and methods for: accelerating and/or augmenting growth in shellfish; improving immunity to diseases and other contaminants; enhancing fertility; and/or increasing the success of larval production and survival.

The subject invention provides methods for improving the health of shellfish through the administration of a cysteamine compound to the shellfish. Specifically, an effective amount of a cysteamine compound is introduced to shellfish to promote shellfish health, growth, and population numbers. For example, cysteamine, or various cysteamine salts, prodrugs, analogs, derivatives, conjugates, and metabolites, are administered to shellfish.

In one embodiment of the invention, a composition comprising a cysteamine compound is introduced into water in which shellfish are harbored. In a related embodiment, the composition comprises additional agents that are useful in promoting shellfish health. For example, antibiotics and/or vaccines may be concurrently administered with a cysteamine compound to shellfish.

The method and composition of the invention are useful for treating shellfish during any stage of development to improve health. A cysteamine compound is preferably administered to shellfish by introducing the cysteamine compound into water that contains or will contain the shellfish to be treated.

### BRIEF DESCRIPTION OF DRAWINGS

FIG. 1 shows a metabolic pathway of cysteamine.

FIG. 2 shows cysteamine as a constituent of co-enzyme A.

### DETAILED DISCLOSURE OF THE INVENTION

The subject invention provides unique materials and methods for improving the health of shellfish. Specifically, the subject invention provides materials and methods for accelerating and/or augmenting growth in shellfish; improving immunity to diseases and other contaminants; enhancing fertility; and/or increasing the success of larval production and survival.

In particular, the invention concerns administering a cysteamine compound to shellfish, in an amount effective to promote shellfish health, growth, and population numbers. One embodiment of the invention is a composition for improving shellfish health, wherein the composition comprises a cysteamine compound. Preferably, the composition is an aqueous mixture or an aqueous emulsion including the cysteamine compound. More preferably, a cysteamine compound (i.e., cysteamine hydrochloride) is provided in solid feed such as sinking or floating feed for shellfish.

As used herein, the term “shellfish” refers to all non-fish aquatic life. As contemplated herein, shellfish includes aquatic invertebrates having a soft, unsegmented body that can be enclosed in a shell. For example, references to shellfish include crustaceans such as prawns, shrimp, crawfish, crayfish, crabs, lobsters; and mollusks such as abalone, clams, mussels, oysters, scallops, octopi, squid, and snails. References to shellfish herein can also include turtles, sea urchins, and sea cucumbers as well as invertebrates that lack a shell (i.e., jellyfish).

The term “shellfish health,” as used herein, generally refers to a variety of parameters that affect the overall condition of a shellfish. Specific parameters upon which shellfish health is based include: the size (or growth) of a shellfish; the length of time in a growing cycle; the immune system's ability to adequately address exposure to diseases and contamination; and the ability to reproduce offspring. As contemplated herein, improving shellfish health includes reducing shellfish mortality; increasing antibody titer/lymphocyte number; and increasing cytokine secretion.

“Concurrent administration” and “concurrently administering,” as used herein, includes administering a compound or method suitable for use with the methods of the invention (administration of a cysteamine compound) to improve shellfish health. For example, an antibiotic and/or vaccine can be administered concurrently with the materials and methods of the invention to improve shellfish health.

According to the subject invention, a compound can be provided in admixture with a cysteamine compound, such as in an aqueous emulsion; or the compound and cysteamine can be provided as separate compounds, such as, for example, separate compositions administered consecutively, simultaneously, or at different times. Preferably, if the cysteamine compound and the known agent (or therapeutic method) for improving shellfish health are administered separately, they are not administered so distant in time from each other that the cysteamine compound and the known agent (method) cannot interact.

Contemplated compounds that can be concurrently administered with a cysteamine compound of the invention include, but are not limited to, Microbicides such as Formalin, Albendazole, Cypermethrin, Deltamethrin, Hydrogen peroxide, and Teflubenzuron; Antimicrobials such as Oxytetracycline, Alkyltrimthylammonium calciumoxytetracycline, Bicozamycin benzoate, cyanphenicol, Doxycycline, Florofenicol, Josamycin, Kitasamycin, Lincomycin, Myroxacin, Nalidixic acid, Phosphomycin, Spiramycin, Sulfadimethoxine-ormetoprim, and Sulfamerazine; and Vaccines such as the Vibrio parahaemolyticus (Vibrogen-S) vaccine, Penaeid multivalent bacterin (P.M.B. vaccine), and Vibrio sp. bacterin.

As used herein, reference to a “cysteamine compound” includes cysteamine, various cysteamine salts (that do not greatly reduce or inhibit the activity of the cysteamine compound), as well as prodrugs of cysteamine that can, for example, be readily metabolized by the shellfish to produce cysteamine endogenously. Also included within the scope of the subject invention are analogs, derivatives, conjugates, and metabolites of cysteamine, which have the ability as, described herein to improve shellfish health. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

As contemplated herein, a cysteamine compound includes pantothenic acid. Pantothenic acid is a naturally occurring vitamin that is converted in mammals to coenzyme A, a substance vital to many mammalian physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

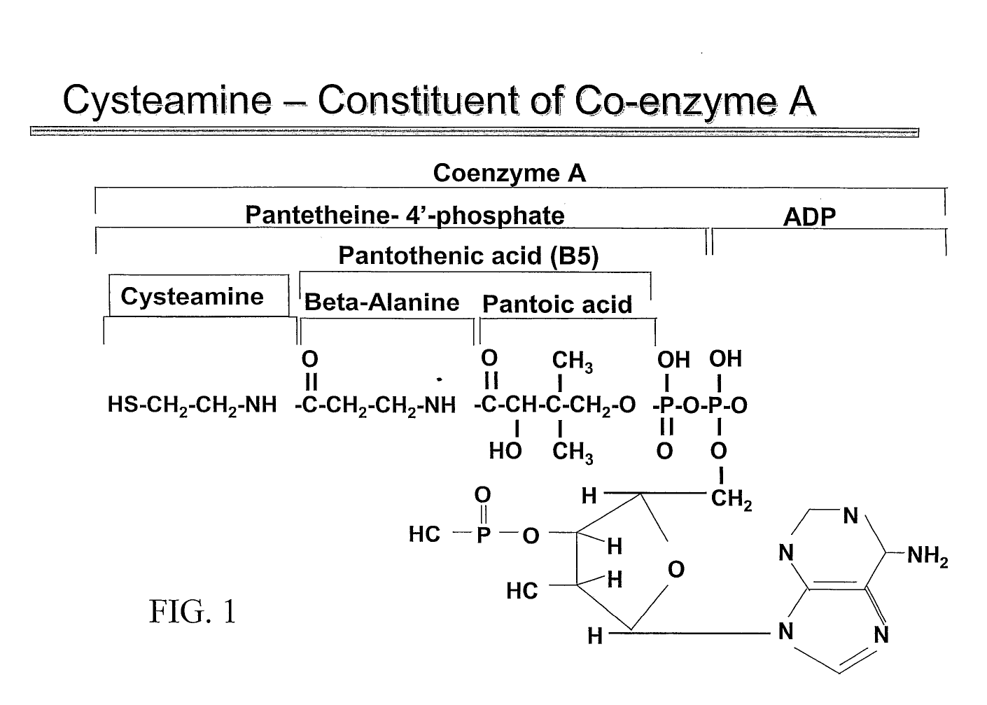

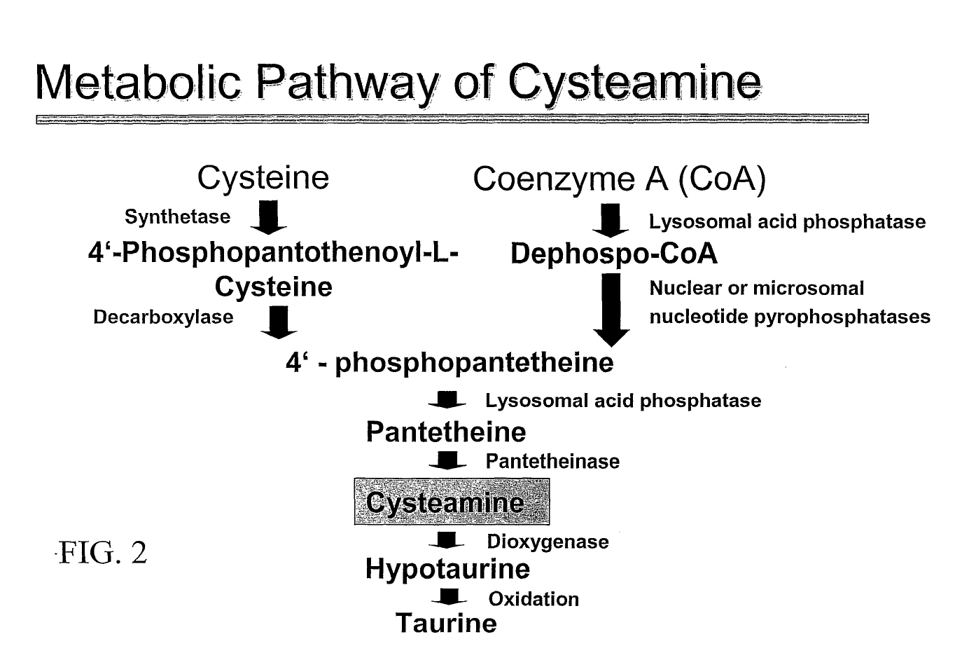

The advantages of cysteamine, as set forth herein, may be achieved in shellfish by promoting the endogenous production of cysteamine through natural metabolic processes such as those observed in mammals (i.e., through the action of co-enzyme A or as a metabolite of cysteine (see FIGS. 1 and 2 of mammalian production of cysteamine)). This may be achieved by, for example, the administration of pantothenic acid to shellfish.

The term “effective amount,” as used herein, refers to the amount necessary to elicit the desired biological response. In accordance with the subject invention, the effective amount of a cysteamine compound is the amount necessary to improve shellfish health. In a preferred embodiment, the effective amount of a cysteamine compound is the amount necessary to accelerate and/or augment growth in shellfish; improve immune response to diseases and other contaminants; enhance fertility; and/or increase the success of larval production and survival. For example, the improvement in shellfish health can be a 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300% acceleration and/or augmentation in growth; improvement in immunity response to a disease and/or contaminant; and/or enhancement in fertility. More specifically, shellfish health is improved as a result of reduced shellfish mortality; increased antibody titer/lymphocyte numbers; and increased cytokine secretion.

Accelerated and/or Augmented Growth

In one embodiment of the invention, a cysteamine compound is administered to shellfish to accelerate and/or augment growth. As contemplated herein, to “accelerate and/or augment growth” refers to the ability to shorten developmental periods during a normal growth cycle and/or increase the overall size of the shellfish.

For example, with oysters, the materials and methods of the invention could shorten the length of time for a normal growth cycle from around 2 years to around 6 months to 1.8 years to reach marketable size. Or, the invention can increase the overall size of an oyster from a cocktail oyster size of 1.5 inches to an appetizer oyster size of about 2.5 inches. In certain embodiments, the subject materials and methods of the invention both accelerate and augment growth (i.e., decrease the length of normal growth cycle and increase overall size of the shellfish).

In another example, the growth cycle of mussels grown in water temperatures below 65° F. takes normally about 2 years to reach market size. By using the materials and methods of the invention, the growth cycle for such shellfish can be decreased from about 2 years to about 6 months to 1.8 years to reach market size.

Improving Immune Response

In another embodiment, a cysteamine compound is administered to shellfish to improve immunity response to diseases and other contaminants. As contemplated herein, “improving immunity” refers to boosting the shellfish immune system to more effectively attack harmful microorganisms and/or contaminants and heal the shellfish of any damages incurred by exposure to such microorganisms and/or contaminants. By improving immunity, the subject invention also increases the likelihood of success in larval production and survival.

According to the subject invention, introduction of a cysteamine compound to shellfish can: (1) proactively augment shellfish immunity to promote resistance to disease and/or contamination; and/or (2) treat and promote rapid recovery from current exposure to harmful microorganisms and/or contamination.

As contemplated herein, the subject invention improves shellfish immune response to a variety of microorganisms and contaminants. For example, the subject invention improves oyster immune response to a variety of diseases and pathogens including, without limitation, Oyster Velar Virus Disease (OVVD); Gill Disease of Portuguese Oysters; Haemocytic Infection Virus Disease of Oysters; Herpes-Type Virus Disease of Oysters; Extracellular Giant “Rickettsiae” of Oysters; Perkinsus marinus (“Dermo” Disease) of Oysters; Perkinsus sp. of European Flat Oysters; Kidney Coccidia of Oysters; Minchinia armoricana of Oysters; Marteiliosis (Aber disease) of Oysters; Marteilioides chungmuensis of Oysters; Bonamia ostreae of Oysters; Oyster Egg Disease; Invasive Ciliates of Juvenile Oysters; Mytilicola intestinalis (Red Worm Disease) of Oysters; Haemocytic Neoplasia of Oysters; Juvenile Disease of Eastern Oysters; Viral Gametocytic Hypertrophy of Oysters; Nocardiosis of Oysters; Mikrocytos machine (Denman Island Disease) of Oysters; Haplosporidium nelsoni (MSX) of Oysters; Haplosporidium costale (SSO) of Oysters; Ostracoblabe implexa (Shell Disease) of Oysters; Oyster Trematode Diseases; Mytilicola orientalis (Red Worm) of Oysters; Parasite Copepods on Oyster Gills; Pea Crabs in Oysters; Malpeque Disease of Oysters; Papova-Like Virus Infection of Pearl Oysters; Apicomplexan Parasite of New Zealand Oysters; Haplosporidium sp. of Pearl Oysters; Marteilia sydneyi of Oysters; Marteilioides branchialis of Oysters; Bonamia exitiosus (Bonamiasis of New Zealand Dredge Oysters); Bonamia sp. (Bonamiasis of Australian Oysters); Mikrocytos roughleyi (Australian Winter Disease) of Oysters; Microsporidiosis of Dredge Oysters; Rikettsia-like and Chlamydia-like Organisms of Oysters; Vibrio spp. (Larval and Juvenile Vibriosis) of Oysters; Hinge Ligament Disease of Juvenile Oysters; Digestive Tract Impaction of Larval Oysters; Gregarine Parasitism of Oysters; Hexamitiasis of Oysters; Ancistrocoina-like Ciliates of Oysters; Sphenophyra-like Ciliates of Oysters; Gill Trichodinids of Oysters; Sirolpidium zoophthorum (Larval Mycosis) of Oysters; Oyster Gill Turbellaria; Nematode Parasitism of Oysters; Shell-boring Polychaetes of Oysters; Shell-burroing Sponges of Oysters; and Pyramidellid Snails of Oysters.

In another example, the subject invention can improve mussel immune response to a variety of diseases and pathogens including, without limitation, Virus-like Diseases of Mussels; Haplosporidian Infection of Mussels; Marteilia refringens/maurini of Mussels; Steinhausia mytilovum (Mussel Egg Disease); Phototrophic Endolity Invasion of Mussel Shells; Proctoeces maculates Trematode Disease of Mussels; Mussel Gill Turbellaria; Mytilicola intestinalis (Red Worm Disease) of Mussels; Kidney Coccidia of Mussels; Bucephalid Trematode Diseases of Mussels; Mytilicola orieiztalis (Red Worm) of Mussels; Pea Crabs in Mussels; Haemocytic Neoplasia of Mussels; Mytilicola porrecta (Red Worm) of Mussels; Rickettsia-like and Chlamydia-like Organisms of Mussels; Gregarine Parasitism of Mussels; Intracellular Ciliates of Mussels; Phenophyra-like Ciliates of Mussels; Ancistrum mytili Gill Ciliate of Mussels; Mycotic Periostracal Sloughing of Mussels; Trematode Metacercariae of Mussels; Parasitic copepods on Mussel Gills; Shell-boring Polychaetes of Mussels; and Shell-burrowing Sponges of Mussels.

In yet another example, the subject invention can improve clam and cockle immune response to a variety of diseases and pathogens including, without limitation, Viral infections of Clams; Brown Ring Disease of Manila Clams; Perkinsus of Clams and Cockles; Haplosporidian Infection of Clams; Microsporidiosis of Clams; Amoeboflagellate Disease of Larval Geoduck Clams; Mytilicola inestinalis (Red Worm Disease) of Clams and Cockles; Nuclear Inclusion X (NIX) of Pacific Razor Clams; Kidney Coccidia of Clams; QPX (quahog parasite unknown) of Clams; Mytilicola orientalis (Red Worm) of Clams and Cockles; Pea Crabs in Clams and Cockles; Haemocytic Neoplasia of Clams; Gonadal Neoplasia of Clams; Endonucleobiotic Bacteria of Clams in Portugal; Mycoplasma-like Infection of Cockles; Cryptosporidiosis of Clams; Marteilia-like Parasite of Giant Clams; Amoebiasis of Cockles; Rickettsia-like and Chlamydia-like Organisms of Clams and Cockles; Vibrio spp. (Larval and Juvenile Vibriosis) of Clams; Hinge Ligament Disease of Juvenile Clams; Gregarine Parasitism of Clams and Cockles; Sphenophyra-like Ciliates of Clams and Cockles, Ancistrocoma pelseneeri and A. myae Ciliates of Clams; Gill Trichodina of Clams and Cockles; Sirolpidium zoophthorum (Larval Mycosis) of Clams; Turbellaria of Clams; Trematode Metacercariae of Clams and Cockles; Shell-boring Polychaetes of Clams; and Siphon Snails of Clams and Cockles.

In a further example, the subject invention can improve scallop immune response to a variety of diseases and pathogens including, without limitation, Perkinsus sp. of Japanese Scallops in Asia; Scallop Haplosporidian; Marteilia sp. of Scallops; Brood-; pouch Copepod of Scallop Gills; Pea Crabs in Scallops; Intracellular Bacterial Disease of Scallops; Bacterial Abscess Lesions of Scallops; Perkinsus qugwadi (SPX) of Scallops; Kidny Coccidia of Scallops; Perdinsus karlssoni of Scallops; Scallop Protistan G; Microsporidiosis of Scallops; Trematode Metacercariae of Scallops; Virus-like Infection of Scallops; Chlamydiosis of Scallops; Rickettsia-like and Chlamydia-like Organisms of Scallops; Vibrio spp. (Larval Vibriosis) of Scallops; Gregarine Parasitism of Scallops; Gill Trichodinids of Scallops; Scallop Gill Turbellaria; Nematode Parastitism of Scallops; Shell-boring Polychaetes of Scallops; and Shell-burrowing Sponges of Scallops.

In another example, the subject invention can improve abalone immune response to a variety of diseases and pathogens including, without limitation, Kidney Coccidia of Abalone; Sabellid Polychaete Infestation of Disease in Abalone; Labyrinthuloides haliotidis of Abalone; Amyotrophia of Abalone; Blister Disease of Cultured Abalone; Withering Syndrome of Abalone; Perkinsus olseni of Abalone; Haplosporidian parasite of Abalone; Fungal Disease of Abalone; Nematode Parasitism of Abalone; Bacterial Diseases of Abalone; Ciliates Associated with Abalone; Trematode Metacercariae of Abalone; and Shell-borring Polychaetes of Abalone.

In yet another example, the subject invention can improve sea urchin immune response to a variety of diseases and pathogens including, without limitation, Namatode Parasitism of Sea Urchin; Bald-Sea-Urchin Disease; Paramoeba invadens of Sea Urchins; Spotted gonad Disease of Sea Urchins; Black Sea Urchin Plage; Trematode Metacercariae of Sea Urchins; and Turbellarian Parasitism of Sea Urchins.

In yet another example, the subject invention can improve lobster immune response to a variety of diseases and pathogens including, without limitation, Paramoeba perniciosa (Paramoebiasis) of Lobsters; Gaffkemia of Lobsters; Anophryoides haemophila (Ciliate Disease) of Lobsters; Pseudocarcinonemetes homari of Lobsters; Microsporidosis of Lobsters; Hematodinium sp. of Norway Lobster; Carcinonemertes australiensis of Lobsters; Parasitic Copepods of Lobsters; vibrio spp. (Juvenile Vibriosis) of Lobsters; Chitinolytic Bacterial Shell Disease of Lobsters; Gregarine Parasitism of Lobsters; Lagenidium sp. (Fungus Disease) of Lobsters; Fusarium sp. (Fungus or Burn Spot Disease) of Lobsters; Haliphthoros sp. (Fungus Disease) of Lobsters; Nematodes in Lobsters; Trematode Metacercariae in Lobsters; and Acanthocephalan Larvae in Lobsters.

In yet another example, the subject invention can improve shrimp and prawn immune response to a variety of diseases and pathogens including, without limitation, Rickettsia-like Infection of Pandalid Shrimp; Protistan Pathogen of Pandalid Shrimp (SPP); Sylon (Rhizocephalan Disease) of Shrimp and Prawns; Baculovirus penaei (BP Virus Disease) of Penaeid Shrimp; Monodon Baculovirus (MBV) Disease of Penaeid Shrimp; Baculoviral Midgut-gland Necrosis (BMN) of Penaeid Shrimp; White Spot Syndrome Baculovirus Complex of Penaeid Shrimp; Hepatopancreatic Parvovirus (HPV) Disease of Shrimp and Prawns; Infectious Hypodermal and Haematopoietic Necrosis Virus (IHHNV) of Penaeid Shrimp; Lymphoidal Parvo-like Virus Disease of Penaeid Shrimp; Lymphoid Organ Vacuolization Virus (LOVV) of Penaeid Shrimp; Reo-like Virus (REO) Disease of Penaeid Shrimp; Taura Syndrome Virus of Penaeid Shrimp; Rhabdovirus Disease of American Penaeid Shrimp; Yellow-head Virus Disease (YHD) of Penaeid Shrimp; Rickettsial Infection of Penaeid Shrimp; Necrotizing Hepatopancreatic of Penaeid Shrimp; Mycobacteriosis of Penaeid Shrimp; Vibrio penaeicida of Cultured Kurama Prawns; Larval Bacterial Necrosis of Freshwater Shrimp; Haplosporidian Infections of Penaeid Shrimp; Gregarine Disease of Penaeid Shrimp; Ciliate Disease of Penaeid Shrimp; Gut and Nerve Syndrom (GNS) of Penaeid Shrimp; Larval Mid-cycle Disease (MCD) of Freshwater Shrimp; Red Disease of Penaeid Shrimp; Vibrio spp. (Vibrio Disease) of Cultured Shrimp; Chitinolytic Bacterial Shell Disease of Shrimp and Prawns; Filamentous Bacterial Disease of Shrimp and Prawns; Microsporidosis (Cotton Shrimp Disease) of Shrimp and Prawns; Larval Mycosis of Shrimp and Prawns; Fusarium sp. (Fungus Disease) of Shrimp and Prawns; Nematomorph Parasitism of Pandalid Shrimp; and Black Gill Syndrome of Shrimp and Prawns.

In yet another example, the subject invention can improve crab immune response to a variety of diseases and pathogens including, without limitation, Viral Diseases of Crabs; Rickettsia and Chlamydia of Crabs; Haplosporidosis of Crabs; Paramoeba perniciosa (Grey Crab Disease); Hematodinium perezi and Hematodinium sp. of Atlantic Crabs; Chitinolytic Fungal Disease (Black Mat Syndrome) of Crabs; Carcinoizemertes spp. of Crabs; Mesanophrys spp. (Ciliate Disease) of Crabs; Hematodinium sp. (Bitter Crab Disease); Rhizocephalan Parasites of Crabs; Hematodinium spp. of Crabs in Australia; Chitinolytic Bacterial Shell Disease of Crabs; Microsporidosis of Crabs; Trematode Metacercariae of Crabs; Acanthocephalan Parasitism of Crabs; Nematomorph Parasitism of Crabs; and Lagendium spp. (Fungus Disease) of Crabs.

In yet another example, the subject invention can improve crayfish immune response to a variety of diseases and pathogens including, without limitation, Psorosperinium spp. (Protozoan Disease) of European Crayfish; Therlohaniasis of Crayfish; Burn Spot Disease (Fungus Disease) of Crayfish; Crayfish Plague (Fungus Disease); Baculovirus of Blue Crayfish; Rickettsia of Crayfish; Nocardia sp. (Bacterial Disease) Crayfish; Proteus or Pseudomonas Bacterial Septicaemia of Crayfish; Chitinolytic Bacterial Shell Disease of Crayfish; Psorospermium sp. (Protozoan Infection) of American Crayfish; Saprolegnia spp. (Fungus Disease) of Crayfish; Trematodes in Crayfish; Turbellaria Infestation of Crayfish; and Branchiobdellida Annelid Parasitism of Crayfish.

Enhancing Fertility

According to the subject invention, a cysteamine compound is administered to shellfish to enhance fertility. As contemplated herein, to “enhance fertility” refers to the ability to maximize fertilization of shellfish. In one embodiment of the subject invention, a cysteamine compound is administered to shellfish to manipulate sexual development. Sexual development manipulation can include increasing the number of eggs and sperm that are produced and discharged by shellfish or shortening the time to which shellfish are capable of reproduction.

Administration of a cysteamine compound to shellfish, in accordance with the subject invention, can be accomplished by any suitable method and technique presently or prospectively known to those skilled in the art. Specifically exemplified herein is the introduction of a cysteamine compound, either alone or concurrently with additional compound(s) or method(s), into water containing the shellfish to be treated. The cysteamine compound can be introduced as a composition, in any available form including in a liquid (i.e., solvent, oil), in an aqueous mixture, in an aqueous emulsion, in a solid carrier or substrate, or other vehicles provided the vehicles are compatible with the administration of the cysteamine compound into water harboring the shellfish to be treated, and do not adversely affect the shellfish.

A variety of suitable adjuvants may also be used in compositions comprising a cysteamine compound. For example, emulsifiers, antifoaming agents (or defoaming agents), antioxidants, preservatives, coloring agents, and the like can be included in compositions of the invention. In one embodiment, the adjuvants are present in compositions of the invention in minor amounts, i.e., less than about 5% by volume, and preferably, less than 1% by volume. In other embodiments, greater amounts of adjuvants are present in compositions of the invention, i.e., up to 70% by volume. All such adjuvants should be noninjurious and nontoxic to shellfish being treated.

According to the present invention, suitable emulsifiers (i.e., surfactants or dispersants) can be cationic, anionic, nonionic, or amphoteric emulsifiers. Preferred emulsifiers include, for example, food grade emulsifiers which are widely available. An overview of some types of suitable emulsifiers for use with the invention include those set forth in A. J. St. Angelo, “A Brief Introduction to Food Emulsion and Emulsifiers,” at pp. 1-8 of G. Charalambous et al., Eds., Food Emulsifiers-Chemistry, Technology, Functional Properties and Applications, Elsevier Science Publishing Co. Inc., New York, N.Y. (1989).

Where needed, after the introduction of a cysteamine compound to water in which shellfish are harbored, a metering or mixing pump, or an inline mixer (i.e., a mixing valve, nozzle or orifice), an aerator, or other device known to the skilled artisan may be used to accomplish the direct dispersion of the cysteamine compound in water.

In one embodiment, an aqueous mixture, emulsion, or dispersion including a cysteamine compound is introduced into water harboring shellfish to be treated. The aqueous mixture, emulsion, or dispersion of the invention can contain from about 0.1% to about 95% of a cysteamine compound, wherein all percentages being by volume, based on the final volume of the composition. The composition can be further diluted when added to the water environment containing the shellfish to be treated according to the present invention. The amount of cysteamine compound used can be varied based upon the health (i.e., size, age, etc.) of the shellfish to be treated.

In a preferred embodiment, a solid feed mixture (i.e., sinking or floating feed) including a cysteamine compound is introduced to shellfish to be treated. The feed mixture of the invention can contain from about 0.1% to about 95% of a cysteamine compound, wherein all percentages being by volume, based on the final volume of the composition. The amount of cysteamine compound used can be varied based upon the health (i.e., size, age, etc.) of the shellfish to be treated.

The cysteamine compounds of the subject invention can be formulated according to known methods for preparing compositions for use in administration to shellfish. The formulations may be presented in unit-dose or multi-dose containers, for example sealed ampoules and vials, and may be stored in a freeze dried (lyophilized) condition requiring only the condition of the sterile liquid carrier, for example, water, prior to use. Extemporaneous solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In certain methods of the invention, the effective amount of cysteamine introduced is dependent on factors such as water pH, hardness, alkalinity, temperature, and the like.

The shellfish that are treated according to the invention include those that are held in a confined body of water, such as a shipping container, holding tank, aquarium, pool, or small pond, large body of water and those that are found in unconfined water, such as streams or off of a beach.

Following is an example that illustrates a procedure for practicing the invention. This example should not be construed as limiting. All percentages are by weight and all solvent mixture proportions are by volume unless otherwise noted.

### EXAMPLE 1

### Shrimp

AQUANIN is a micro-granule, produced by the Walcom Bio-Chemical Company, Shanghai, China, 30% of which is comprised of cysteamine hydrochloride as an active ingredient for livestock application. Example 1 describes the effect of AQUANIN on juvenile shrimp (Penaeus vannamei), in particular the effect of cysteamine hydrochloride on shrimp growth in different dosages.

Juvenile shrimp (Penaeus vannamei) with average body weight 0.43-0.49 g were used in this example. The juvenile shrimp were fed standard shrimp feed provided from Guangdong ZhanJiang HengXing company in Guangdong, China. The shrimp feed was composed of the following products listed in Table 1.

TABLE 1
Shrimp Feed

| | |
|---|---|
| Fishmeal | 35% |
| Fermented soybean meal | 28% |
| Peanut meal | 5% |
| Wheat flour | 24.885% |
| Phosphor fat meal | 2% |
| Fish oil | 1% |
| Dicalcium phosphate | 1% |
| Maltose | 2% |
| Vitamin premix | 0.1% |
| Mineral premix | 0.1% |
| Choline Chloride | 0.5% |
| Antioxidant | 0.015% |

The trial experiment of Example 1 was conducted for 42 days. A total of 30 tails of juvenile shrimp with average body weights of 0.43-0.49 g were randomly divided into 4 groups (G1, G2, G3 and a Control group), each group contained 3 replicates, each replicate used one aquarium (size: 0.8×0.6×0.6 meters), with sea water circulation. The G1 group was fed 500 ppm AQUANIN (150 ppm of cysteamine hydrochloride) and standard shrimp feed. The G2 group was fed 1,000 ppm AQUANIN (300 ppm cysteamine hydrochloride) and standard shrimp feed. The G3 group was fed 3,000 ppm AQUANIN (900 ppm cysteamine hydrochloride) and standard shrimp feed. The Control group was fed standard shrimp feed only.

During the trial period, circulation of water was maintained, and water quality was kept at normal. At the end of this experiment, the total number of shrimp, the total body weight of the shrimp, and the average body weight of each shrimp were calculated.

Calculation of absolute body weight gain was performed by taking the average final body weight of each shrimp and subtracting this value from the average initial body weight of each shrimp.

Calculation of relative body weight gain was performed by dividing the average absolute body weight gain by the average initial body weight and multiplying this value by 100.

In the following Table 2, the data suggests that 500 ppm of AQUANIN presented the best growth promoting effect when compared against the other groups. The G1 group demonstrated: that 90% of the shrimp were alive at the end of the trial session, 11.95% increase in absolute body weight gain, and 6.25% improvement in food conversion ratio (FCR) after 42 days of treatment. The G2 and G3 groups, which were administered 1,000 ppm and 3,000 ppm of AQUANIN, respectively, also demonstrated some growth within the 42 day trial period. Specifically, the G2 and G3 groups demonstrated a 7.54-8.45% increase in absolute body weight gain and a 3.41-4.54% improvement in FCR.

TABLE 2
Effect of AQUANIN on Juvenile Shrimp (Penaeus vannamei) Growth.

| | 500 ppm | 1,000 ppm | 3,000 ppm | |
|---|---|---|---|---|
| | Aquanin | Aquanin | Aquanin | Control |
| No. of shrimp | 90 | 90 | 90 | 90 |
| Average initial body | 0.47 ± 0.02 | 0.46 ± 0.02 | 0.46 ± 0.02 | 0.452 ± 0.02 |
| weight (g/shrimp) | | | | |
| Average final body | 4.03 ± 0.18 | 3.91 ± 0.22 | 3.88 ± 0.66 | 3.63 ± 0.28 |
| weight (g/shrimp) | | | | |
| Absolute body weight | 3.56 ± 0.16 | 3.45 ± 0.19 | 3.42 ± 0.54 | 3.18 ± 0.24 |
| gain(g/shrimp) | | | | |
| Improvement in absolute | 11.95 | 8.45 | 7.54 | — |
| body weight gain (%) | | | | |
| Relative body weight | 773.80 ± 54.03 | 749.65 ± 18.36 | 749.24 ± 40.85 | 710.18 ± 70.09 |
| gain (%) | | | | |
| Improvement in relative | 7.46 | 5.49 | 5.48 | — |
| body weight gain (%) | | | | |
| Feed Conversion Ratio | 1.65 ± 0.09 | 1.70 ± 0.09 | 1.68 ± 0.06 | 1.76 ± 0.11 |
| (FCR) | | | | |
| Improvement in FCR (%) | 6.25 | 3.41 | 4.54 | — |
| Average live shrimp (%) | 90.00 ± 9.81 | 77.50 ± 11.67 | 89.17 ± 5.69 | 87.22 ± 10.63 |

As illustrated in Table 2, the addition of 500 ppm AQUANIN produced the best growth promoting effect on the juvenile shrimp. Thus, the administration of a cysteamine compound, in accordance with the subject invention, can help promote shellfish (in particular shrimp) health, including promotion of shellfish growth, improvement of shellfish feed conversion, enhancing shellfish longevity, and increasing shellfish body weight.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for improving shellfish health, wherein said method comprises administering to shellfish an effective amount of a cysteamine compound and shellfish feed.

2. The method of claim 1, wherein the shellfish is shrimp and the shellfish feed is shrimp feed.

3. The method of claim 2, wherein the effective amount of cysteamine compound is 150 ppm of cysteamine hydrochloride.

4. The method of claim 1, wherein the shellfish is selected from the group consisting of: prawns, shrimp, crawfish, crayfish, crabs, lobsters, abalone, clams, mussels, oysters, scallops, octopi, squid, and snails.

5. The method of claim 1, wherein the shellfish is selected from the group consisting of turtles, sea urchins, sea cucumbers, and jellyfish.

6. The method of claim 1, further comprising the step of concurrently administering the cysteamine compound with other known compounds used to improve shellfish health.

7. The method of claim 6, wherein the other known compounds are selected from the group consisting of: microbicides, antimicrobials, and vaccines.

8. The method of claim 7, wherein the other known compounds are selected from the group consisting of: Formalin, Albendazole, Cypermethrin, Deltamethrin, Hydrogen peroxide, Teflubenzuron, Oxytetracycline, Alkyltrimthylammonium calciumoxytetracycline, Bicozamycin benzoate, cyanphenicol, Doxycycline, Florofenicol, Josamycin, Kitasamycin, Lincomycin, Myroxacin, Nalidixic acid, Phosphomycin, Spiramycin, Sulfadimethoxine-ormetoprim, Sulfamerazine, Vibrio parahaemolyticus (Vibrogen-S) vaccine, Penaeid multivalent bacterin (P.M.B. vaccine), and Vibrio sp. bacterin.

9. The method of claim 1, the administration of the cysteamine compound improves shellfish immune response or enhances fertility.

10. The method of claim 9, wherein the shellfish is oyster and the improved immune response is to diseases and pathogens selected from the group consisting of: Oyster Velar Virus Disease (OVVD); Gill Disease of Portuguese Oysters; Haemocytic Infection Virus Disease of Oysters; Herpes-Type Virus Disease of Oysters; Extracellular Giant “Rickettsiae” of Oysters; Perkinsus marinus (“Dermo” Disease) of Oysters; Perkinsus sp. of European Flat Oysters; Kidney Coccidia of Oysters; Minchinia armoricana of Oysters; Marteiliosis (Aber disease) of Oysters; Marteilioides chungmuensis of Oysters, Bonamia ostreae of Oysters; Oyster Egg Disease; Invasive Ciliates of Juvenile Oysters; Mytilicola intestinalis (Red Worm Disease) of Oysters; Haemocytic Neoplasia of Oysters; Juvenile Disease of Eastern Oysters; Viral Gametocytic Hypertrophy of Oysters; Nocardiosis of Oysters; Mikrocytos machine (Denman Island Disease) of Oysters; Haplosporidium nelsoni (MSX) of Oysters; Haplosporidium costale (SSO) of Oysters; Ostracoblabe implexa (Shell Disease) of Oysters; Oyster Trematode Diseases; Mytilicola orientalis (Red Worm) of Oysters; Parasite Copepods on Oyster Gills; Pea Crabs in Oysters; Malpeque Disease of Oysters; Papova-Like Virus Infection of Pearl Oysters; Apicomplexan Parasite of New Zealand Oysters; Haplosporidium sp. of Pearl Oysters; Marteilia sydneyi of Oysters; Marteilioides branchialis of Oysters; Bonamia exitiosus (Bonamiasis of New Zealand Dredge Oysters); Bonamia sp. (Bonamiasis of Australian Oysters); Mikrocytos roughleyi (Australian Winter Disease) of Oysters; Microsporidiosis of Dredge Oysters; Rikettsia-like and Chlamydia-like Organisms of Oysters; Vibrio spp. (Larval and Juvenile Vibriosis) of Oysters; Hinge Ligament Disease of Juvenile Oysters; Digestive Tract Impaction of Larval Oysters; Gregarine Parasitism of Oysters; Hexamitiasis of Oysters; Ancistrocoma-like Ciliates of Oysters; Sphenophyra-like Ciliates of Oysters; Gill Trichodinids of Oysters; Sirolpidium zoophthorum (Larval Mycosis) of Oysters; Oyster Gill Turbellaria; Nematode Parasitism of Oysters; Shell-boring Polychaetes of Oysters; Shell-burroing Sponges of Oysters; and Pyramidellid Snails of Oysters

11. The method of claim 9, wherein the shellfish is mussel and the improved immune response is to diseases and pathogens selected from the group consisting of: Virus-like Diseases of Mussels; Haplosporidian Infection of Mussels; Marteilia refringens/maurini of Mussels; Steinhausia mytilovum (Mussel Egg Disease); Phototrophic Endolity Invasion of Mussel Shells; Proctoeces maculates Trematode Disease of Mussels; Mussel Gill Turbellaria; Mytilicola intestinalis (Red Worm Disease) of Mussels; Kidney Coccidia of Mussels; Bucephalid Trematode Diseases of Mussels; Mytilicola orientalis (Red Worm) of Mussels; Pea Crabs in Mussels; Haemocytic Neoplasia of Mussels; Mytilicola porrecta (Red Worm) of Mussels; Rickettsia-like and Chlamydia-like Organisms of Mussels; Gregarine Parasitism of Mussels; Intracellular Ciliates of Mussels; Phenophyra-like Ciliates of Mussels; Ancistrum mytili Gill Ciliate of Mussels; Mycotic Periostracal Sloughing of Mussels; Trematode Metacercariae of Mussels; Parasitic copepods on Mussel Gills; Shell-boring Polychaetes of Mussels; and Shell-burrowing Sponges of Mussels.

12. The method of claim 9, wherein the shellfish are clam and cockle and the improved immune response is to diseases and pathogens selected from the group consisting of: Viral infections of Clams; Brown Ring Disease of Manila Clams; Perkinsus of Clams and Cockles; Haplosporidian Infection of Clams; Microsporidiosis of Clams; Amoeboflagellate Disease of Larval Geoduck Clams; Mytilicola inestinalis (Red Worm Disease) of Clams and Cockles; Nuclear Inclusion X (NIX) of Pacific Razor Clams; Kidney Coccidia of Clams; QPX (quahog parasite unknown) of Clams; Mytilicola orientalis (Red Worm) of Clams and Cockles; Pea Crabs in Clams and Cockles; Haemocytic Neoplasia of Clams; Gonadal Neoplasia of Clams; Endonucleobiotic Bacteria of Clams in Portugal; Mycoplasma-like Infection of Cockles; Cryptosporidiosis of Clams; Marteilia-like Parasite of Giant Clams; Amoebiasis of Cockles; Rickettsia-like and Chlamydia-like Organisms of Clams and Cockles; Vibrio spp. (Larval and Juvenile Vibriosis) of Clams; Hinge Ligament Disease of Juvenile Clams; Gregarine Parasitism of Clams and Cockles; Sphenophyra-like Ciliates of Clams and Cockles, Ancistrocoma pelseneeri and A. myae Ciliates of Clams; Gill Trichodina of Clams and Cockles; Sirolpidium zoophthorum (Larval Mycosis) of Clams; Turbellaria of Clams; Trematode Metacercariae of Clams and Cockles; Shell-boring Polychaetes of Clams; and Siphon Snails of Clams and Cockles.

13. The method of claim 9, wherein the shellfish is scallop and the improved immune response is to diseases and pathogens selected from the group consisting of: Perkinsus sp. of Japanese Scallops in Asia; Scallop Haplosporidian; Marteilia sp. of Scallops; Brood-; pouch Copepod of Scallop Gills; Pea Crabs in Scallops; Intracellular Bacterial Disease of Scallops; Bacterial Abscess Lesions of Scallops; Perkinsus qugwadi (SPX) of Scallops; Kidny Coccidia of Scallops; Perdinsus karlssoni of Scallops; Scallop Protistan G; Microsporidiosis of Scallops; Trematode Metacercariae of Scallops; Virus-like Infection of Scallops; Chlamydiosis of Scallops; Rickettsia-like and Chlamydia-like Organisms of Scallops; Vibrio spp. (Larval Vibriosis) of Scallops; Gregarine Parasitism of Scallops; Gill Trichodinids of Scallops; Scallop Gill Turbellaria; Nematode Parastitism of Scallops; Shell-boring Polychaetes of Scallops; and Shell-burrowing Sponges of Scallops.

14. The method of claim 9, wherein the shellfish is abalone and the improved immune response is to diseases and pathogens selected from the group consisting of: Kidney Coccidia of Abalone; Sabellid Polychaete Infestation of Disease in Abalone; Labyrinthuloides haliotidis of Abalone; Amyotrophia of Abalone; Blister Disease of Cultured Abalone; Withering Syndrome of Abalone; Perkinsus olseni of Abalone; Haplosporidian parasite of Abalone; Fungal Disease of Abalone; Nematode Parasitism of Abalone; Bacterial Diseases of Abalone; Ciliates Associated with Abalone; Trematode Metacercariae of Abalone; and Shell-borring Polychaetes of Abalone.

15. The method of claim 9, wherein the shellfish is sea urchin and the improved immune response is to diseases and pathogens selected from the group consisting of: Namatode Parasitism of Sea Urchin; Bald-Sea-Urchin Disease; Paramoeba invadens of Sea Urchins; Spotted gonad Disease of Sea Urchins; Black Sea Urchin Plage; Trematode Metacercariae of Sea Urchins; and Turbellarian Parasitism of Sea Urchins.

16. The method of claim 9, wherein the shellfish is lobster and the improved immune response is to diseases and pathogens selected from the group consisting of: Paramoeba perniciosa (Paramoebiasis) of Lobsters; Gaffkemia of Lobsters; Anophryoides haemophila (Ciliate Disease) of Lobsters; Pseudocarcinonemetes homari of Lobsters; Microsporidosis of Lobsters; Hematodinium sp. of Norway Lobster; Carcinonemertes australiensis of Lobsters; Parasitic Copepods of Lobsters; vibrio spp. (Juvenile Vibriosis) of Lobsters; Chitinolytic Bacterial Shell Disease of Lobsters; Gregarine Parasitism of Lobsters; Lagenidium sp. (Fungus Disease) of Lobsters; Fusarium sp. (Fungus or Burn Spot Disease) of Lobsters; Haliphthoros sp. (Fungus Disease) of Lobsters; Nematodes in Lobsters; Trematode Metacercariae in Lobsters; and Acanthocephalan Larvae in Lobsters.

17. The method of claim 9, wherein the shellfish are shrimp and prawn and the improved immune response is to diseases and pathogens selected from the group consisting of: Rickettsia-like Infection of Pandalid Shrimp; Protistan Pathogen of Pandalid Shrimp (SPP); Sylon (Rhizocephalan Disease) of Shrimp and Prawns; Baculovirus penaei (BP Virus Disease) of Penaeid Shrimp; Monodon Baculovirus (MBV) Disease of Penaeid Shrimp; Baculoviral Midgut-gland Necrosis (BMN) of Penaeid Shrimp; White Spot Syndrome Baculovirus Complex of Penaeid Shrimp; Hepatopancreatic Parvovirus (HPV) Disease of Shrimp and Prawns; Infectious Hypodermal and Haematopoietic Necrosis Virus (IHHNV) of Penaeid Shrimp; Lymphoidal Parvo-like Virus Disease of Penaeid Shrimp; Lymphoid Organ Vacuolization Virus (LOVV) of Penaeid Shrimp; Reo-like Virus (REO) Disease of Penaeid Shrimp; Taura Syndrome Virus of Penaeid Shrimp; Rhabdovirus Disease of American Penaeid Shrimp; Yellow-head Virus Disease (YHD) of Penaeid Shrimp; Rickettsial Infection of Penaeid Shrimp; Necrotizing Hepatopancreatic of Penaeid Shrimp; Mycobacteriosis of Penaeid Shrimp; Vibrio penaeicida of Cultured Kurama Prawns; Larval Bacterial Necrosis of Freshwater Shrimp; Haplosporidian Infections of Penaeid Shrimp; Gregarine Disease of Penaeid Shrimp; Ciliate Disease of Penaeid Shrimp; Gut and Nerve Syndrom (GNS) of Penaeid Shrimp; Larval Mid-cycle Disease (MCD) of Freshwater Shrimp; Red Disease of Penaeid Shrimp; Vibrio spp. (Vibrio Disease) of Cultured Shrimp; Chitinolytic Bacterial Shell Disease of Shrimp and Prawns; Filamentous Bacterial Disease of Shrimp and Prawns; Microsporidosis (Cotton Shrimp Disease) of Shrimp and Prawns; Larval Mycosis of Shrimp and Prawns; Fusarium sp. (Fungus Disease) of Shrimp and Prawns; Nematomorph Parasitism of Pandalid Shrimp; and Black Gill Syndrome of Shrimp and Prawns.

18. The method of claim 9, wherein the shellfish is crab and the improved immune response is to diseases and pathogens selected from the group consisting of: Viral Diseases of Crabs; Rickettsia and Chlamydia of Crabs; Haplosporidosis of Crabs; Paramoeba perniciosa (Grey Crab Disease); Hematodinium perezi and Hematodinium sp. of Atlantic Crabs; Chitinolytic Fungal Disease (Black Mat Syndrome) of Crabs; Carcinonemertes spp. of Crabs; Mesanophrys spp. (Ciliate Disease) of Crabs; Hematodinium sp. (Bitter Crab Disease); Rhizocephalan Parasites of Crabs; Hematodinium spp. of Crabs in Australia; Chitinolytic Bacterial Shell Disease of Crabs; Microsporidosis of Crabs; Trematode Metacercariae of Crabs; Acanthocephalan Parasitism of Crabs; Nematomorph Parasitism of Crabs; and Lagendium spp. (Fungus Disease) of Crabs.

19. The method of claim 9, wherein the shellfish is crayfish and the improved immune response is to diseases and pathogens selected from the group consisting of: Psorosperinium spp. (Protozoan Disease) of European Crayfish; Therlohaniasis of Crayfish; Burn Spot Disease (Fungus Disease) of Crayfish; Crayfish Plague (Fungus Disease); Baculovirus of Blue Crayfish; Rickettsia of Crayfish; Nocardia sp. (Bacterial Disease) Crayfish; Proteus or Pseudomonas Bacterial Septicaemia of Crayfish; Chitinolytic Bacterial Shell Disease of Crayfish; Psorosperinium sp. (Protozoan Infection) of American Crayfish; Saprolegnia spp. (Fungus Disease) of Crayfish; Trematodes in Crayfish; Turbellaria Infestation of Crayfish; and Branchiobdellida Annelid Parasitism of Crayfish.

20. A composition comprising an effective amount of a cysteamine compound for improving shellfish health and a shellfish feed.

21. The composition of claim 20, wherein the shellfish feed is shrimp feed.

22. The composition of claim 20, wherein the composition is provided in a form selected from the group consisting of: liquid, aqueous mixture, aqueous emulstion, solid carrier, and solid substrate.

23. The composition of claim 20, further comprising about 0.1% to 95% of the cysteamine compound.
